(19) United States
(12) Patent Application Publication
(10) Pub. No.: US 2023/0277828 A1
(43) Pub. Date: Sep. 7, 2023

(54) MICRONEEDLE CASTING SYSTEM AND MICRONEEDLE FABRICATION METHOD

(71) Applicant: Suzhou Rêveda Medical Co., Ltd., Shanghai (CN)

(72) Inventors: Long Liu, Shanghai (CN); Ping Yan, Shanghai (CN); Yuan Huang, Shanghai (CN); Qiuyu Qu, Shanghai (CN)

(73) Assignee: Suzhou Rêveda Medical Co., Ltd., Shanghai (CN)

(21) Appl. No.: 18/008,360

(22) PCT Filed: Jun. 22, 2020
(86) PCT No.: PCT/CN2020/097466

§ 371 Date: Dec. 5, 2022

(30) Foreign Application Priority Data
Jun. 16, 2020 (CN) 202010549505.9

(51) Int. Cl. A61M 37/00

(52) U.S. Cl. CPC A61M 37/0015; A61M 2037/0053; A61M 2037/0061

(57) ABSTRACT

A microneedle casting system includes a vacuum chamber, a motion platform, a first motion assembly, a liquid-filling needle assembly, a second motion assembly, and a controller. The motion platform is arranged in the vacuum chamber; the first motion assembly includes a first transmission component and a first drive component that are connected to each other; the liquid-filling needle assembly includes a liquid-dispensing tip and a liquid-filling needle shaft; the second motion assembly includes a second transmission component and a second drive component which are connected to each other, the liquid-filling needle is connected to the second transmission component, and, driven by the second drive component, the second transmission component drives the liquid-filling needle to move in a second direction; and the first drive component or/and the second drive component are communicatively connected to the controller.

### TECHNICAL FIELD

The present invention relates to the technical field of microneedles, and more particularly, to a microneedle casting system and a microneedle fabrication method.

### BACKGROUND ART

Micro-molding is a high-precision micro-nano fabrication technique that shapes microstructures employing microreplication molds. This technique has the advantages of high replication precision, low cost, and low residual stress, and is widely used for the fabrication of micro-nano structures such as micro-gears, microneedles, micro-fluidic chips, and light guide plates in mechanical, medical, and biological fields. The most critical step in micro-molding is mold filling, that is, filling a recess of a mold with a replication liquid material at a high filling ratio, which is a key factor affecting the accuracy of the microstructure replication.

The conventional mold filling methods include pressure filling, centrifugal filling, and vacuum filling. In pressure filling, the filling material is pressed into a recess in a mold. It is difficult to process a mold that features a high depth-to-width ratio and a high-precision structure, such as a recess of a microneedle, through the method of metal integral forming. Therefore, molds with a micro-recess are often made of silicon materials or polymeric materials. However, the silicon molds with a micro-recess are relatively fragile, and the polymer molds with a micro-recess (e.g., PDMS and SU-8 glue molds) are soft in texture, imposing a high requirement on the embossing force, hence they are not suitable for mass production.

In contrast, vacuum filling has lower requirements for mold material and size compatibility and is thus more advantageous. In the vacuum filling method for microneedles in the prior art, a casting solution is applied evenly on the surface of the mold at normal pressure, the mold is evacuated to remove the residual gas inside the mold, and the casting solution enters the microstructure of the mold to complete the filling replication.

Chinese patent publication No. CN106426687A mentions a vacuum-filling device for filling a microneedle mold with a high-viscosity liquid. With the device, firstly, the casting solution is applied evenly, and then a certain negative pressure state is maintained on a back surface of a microneedle mold, so that the high-viscosity liquid flows into an inverted cone pattern of the microneedle to achieve a better mold filling effect. However, in this method, the material of the microneedle mold must have good liquid barrier properties and the mold must not be too thick, otherwise, the residual gas in the mold will be prevented from being withdrawn from the back of the mold, leading to an inferior filling quality of the mold. Moreover, if the negative pressure is not evenly distributed on the back of the mold, the consistency in filling the mold will be even worse. In addition, it is difficult to ensure that the components of the solution do not enter the interior of the mold material at all, and if any polymer (drug) in the solution penetrates the mold, the original characteristics of the mold material will be altered, which reduces the repeated service life of the mold.

Chinese patent publication No. CN110582320A mentions a method of manufacturing a microneedle patch. In the method, a liquid barrier permeable material is also introduced to make a mold, that is, before use, firstly, the mold is evacuated for a while to remove the gas inside the mold, and then the mold is filled with a casting liquid; the microneedle cavity is filled by taking advantage of the backflow of the gas inside the mold. This method has special requirements for the mold material, and it is difficult to ensure a quantifiable consistency in different parts of one mold and between different molds when the evacuated mold is cast under normal pressure and the gas inside the mold flows back. The whole casting process, including the evacuation of the mold in the early stage and the backflow of gas after filling, is relatively long, which is not conducive to mass production.

A new microneedle casting system and microneedle fabrication method are desirable to address one or more of the problems described above in the art.

### SUMMARY OF THE INVENTION

The technical problem to be solved by the present invention is how to provide a microneedle casting system and microneedle fabrication method that can rapidly complete uniform casting of a large-area planar microneedle casting mold, render high-precision and rapid replication of a micro-nano structure, and have the advantages of less consumption of a casting solution, accurate control, high casting efficiency, good consistency, and greatly reduced costs.

To solve the above technical problem, the present invention provides a microneedle casting system, including: a vacuum chamber, a motion platform, a first motion assembly, a liquid-filling needle assembly, a second motion assembly, and a controller; wherein the motion platform is arranged in the vacuum chamber for supporting a microneedle casting mold; the first motion assembly includes a first transmission component and a first drive component that are connected to each other, the motion platform is connected to the first transmission component, and the first transmission component, driven by the first drive component, moves the motion platform in a first direction or/and a third direction; the liquid-filling needle assembly is configured to convey a casting solution for fabricating the microneedle into the vacuum chamber, the liquid-filling needle assembly includes a liquid-dispensing tip and a liquid-filling needle shaft, one end of the liquid-filling needle shaft extends into the vacuum chamber and is connected to the liquid-dispensing tip; the second motion assembly includes a second transmission component and a second drive component that are connected to each other, the liquid-filling needle shaft is connected to the second transmission component, and the second transmission component, driven by the second drive component, moves the liquid-filling needle assembly in a second direction; the controller is communicatively connected to the first drive component and the second drive component, respectively, the controller is configured, when the vacuum chamber is vacuum, to control the second drive component to drive the liquid-filling needle assembly to move closer to the motion platform in the second direction and to control the liquid-filling needle assembly to output the casting solution for fabricating the microneedle, and to control the first drive component to drive the motion platform to move in the first direction and/or the third direction; wherein the first direction, the second direction and the third direction are perpendicular to each other.

Preferably, the first drive component is a first motor, the first motor having an output end connected to the first transmission component; the first transmission component includes a support frame, a guide rod, a first screw, and a moving member, where the first screw is rotatably arranged on the support frame in the first direction, the guide rod is arranged in the first direction and passes through the moving member, the moving member is in a threaded connection with the first screw and moves in the first direction under the action of the guide rod and the first screw, and the motion platform is fixedly connected to the moving member.

Preferably, the first drive component is outside the vacuum chamber, the first transmission component is inside the vacuum chamber, and the first drive component and the first transmission component are connected through a connecting mechanism; the connecting mechanism includes a connecting shaft, a first coupling, and a second coupling, where one end of the connecting shaft is connected to an output end of the first motor via the first coupling, and the other end of the connecting shaft passes through a side wall of the vacuum chamber and the support frame and is connected to the first screw through the second coupling; the connecting shaft is sealed and rotatably arranged on the side wall of the vacuum chamber between the first drive component and the first transmission component.

Preferably, the first drive component is a first motor, the first motor having an output end connected to the first transmission component; the first transmission component includes a support frame, a second screw, a third screw, and a moving member, where the second screw and the third screw are rotatably arranged on the support frame in the first direction, the moving member moves in the first direction by being threadedly connected to the second screw and the third screw, and the motion platform is fixedly connected to the moving member.

Preferably, the second screw has a second thread for a threaded connection with the moving member, the third screw has a third thread for a threaded connection with the moving member, the second screw and the third screw are driven to rotate in one direction, and the second thread has an identical pitch and direction to those of the third thread; alternatively,

the second screw and the third screw are driven to rotate in opposite directions, and the second thread has an identical pitch to but a different direction from that of the third thread.

Preferably, the first drive component is outside the vacuum chamber, the first transmission component is inside the vacuum chamber, and the first drive component and the first transmission component are connected through a connecting mechanism; the connecting mechanism includes a connecting shaft and a connecting gear set, one end of the connecting shaft is connected to an output shaft of the first motor, the other end of the connecting shaft passes through a side wall of the vacuum chamber and the support frame and is connected to the second screw and the third screw through the connecting gear set; the connecting shaft is sealed and rotatably arranged on the side wall of the vacuum chamber between the first drive component and the first transmission component.

Preferably, a bearing and a sealing ring are provided between the connecting shaft and the side wall of the vacuum chamber between the first drive component and the first transmission component, so that the connecting shaft is sealed and rotatably arranged on the side wall of the vacuum chamber between the first drive component and the first transmission component.

Preferably, the second drive component is a second motor, and the second transmission component includes a vertical post, a sliding rail, and a slider, wherein the vertical post is fixedly arranged outside the vacuum chamber, the sliding rail is arranged on the vertical post in the second direction, one side of the slider is movably connected to the sliding rail, and the other side of the slider is fixedly connected to the liquid-filling needle shaft; the sliding rail, driven by the second motor, moves the liquid-filling needle assembly in the second direction.

Preferably, the liquid-filling needle assembly further includes a pressure-reducing valve within the liquid-filling needle shaft for performing one or more functions of pressure reducing, pressure stabilizing, and back-flowing of the casting solution.

Preferably, the relief valve includes a valve body, an inner cavity is formed in the valve body, a first spool valve and a second spool valve are provided in the inner cavity at an interval axially, and the first spool valve and the second spool valve are movable relative to the inner cavity; an inflow channel, an inflow hole, an outflow hole, and an outflow channel are provided on the valve body, the inflow channel and the outflow channel are blind holes, and the inflow channel and the outflow channel extend axially on the valve body; the inflow channel is in communication with the inner cavity through the inflow hole, the outflow channel is in communication with the inner cavity through the outflow hole, and according to positions of the inflow hole and the outflow hole, the inner cavity is divided into a first cavity, a cavity channel, and a second cavity successively; the first spool valve is configured to, initially, hold at a first position under a first elastic force and abut against the inflow hole to cut off communication between the cavity channel and the inflow channel; move axially from the first position to the first cavity against the first elastic force under a first axial pressure of the casting solution greater than the first elastic force to communicate the cavity channel with the inflow channel; the second spool valve is configured to, initially, hold in a second position under a second elastic force to cut off communication between the cavity channel between the first spool valve and the second spool valve and the outflow channel; move axially along the inner cavity from the second position to the second cavity against the second elastic force under a second axial pressure of the casting solution greater than the second elastic force, to communicate the cavity channel between the first spool valve and the second spool valve with the outflow channel.

Preferably, the first spool valve is further configured to return to the first position under the first elastic force when the first axial pressure of the casting solution is less than the first elastic force or the first axial pressure of the casting solution is removed; the second spool valve is further configured to return to the second position under the second elastic force when the second axial pressure of the casting solution is less than the second elastic force or the second axial pressure of the casting solution is removed.

Preferably, a cavity wall of the first cavity is further provided with a damping hole through which the outflow channel is communicated with the first cavity.

Preferably, the pressure-reducing valve further includes a first secured member and a second secured member, the inner cavity has a third end and a fourth end, the first secured member is secured to an end of the third end of the inner cavity, and the second secured member is secured to an end of the fourth end of the inner cavity; a first elastic structure is provided in the first cavity for providing the first elastic force, one end of the first elastic structure abuts the first spool valve, and the other end of the first elastic structure abuts the first secured member; a second elastic structure is arranged within the second cavity for providing the second elastic force, one end of the second elastic structure abuts the second spool valve, and the other end of the second elastic structure abuts the second secured member.

Preferably, the first spool valve includes a first spool valve body having a hollow first stop post extending axially within the first cavity, and the first elastic structure is arranged within the first stop post; the second spool valve includes a second spool valve body having a hollow second stop post extending axially within the second cavity, and the second elastic structure is arranged within the second stop post.

Preferably, the first secured member is further provided with a first recess for receiving the first stop post; the second secured member is further provided with a second recess for receiving the second stop post.

Preferably, a first projection is formed by extending the first recess towards the first spool valve, and the other end of the first elastic structure is sheathed outside the first projection; a second projection is formed by extending the second recess towards the second spool valve, and the other end of the second elastic structure is sheathed outside the second projection.

Preferably, a cavity wall of the first cavity is further provided with a damping hole through which the outflow channel is communicated with the first cavity;

a distance between the damping hole and the first position is greater than a distance between an open end of the first stop post and a bottom of the first recess.

Preferably, the valve body is provided with a first valve seat and a second valve seat, wherein the first valve seat is configured to maintain the first spool valve at the first position and prevent the first spool valve from approaching the second spool valve, and the second valve seat is configured to maintain the second spool valve at the second position and prevent the second spool valve from approaching the first spool valve.

Preferably, the valve body is further provided with a back-flow hole through which the outflow channel is communicated with the inner cavity, the back-flow hole being located between the outflow hole and an outlet of the outflow channel.

Preferably, the first spool valve is a piston and the second spool valve is a diaphragm, the piston having a ramp for generating the first axial pressure of the casting solution against the piston.

Preferably, the liquid-filling needle assembly further includes a filling pump, one end of the liquid-filling needle shaft away from an end of the liquid-dispensing tip being connected to the filling pump, the filling pump being communicatively connected to the controller.

Preferably, the system further includes a mixing tank connected to the filling pump for mixing various raw materials for fabricating the microneedle uniformly to form the casting solution for fabricating the microneedle.

Preferably, the other end of the liquid-filling needle shaft is connected to a pressure relief valve, and the pressure relief valve is connected to the liquid-filling needle shaft and the filling pump through hoses, respectively, for relieving liquid pressure in the liquid-filling needle shaft.

Preferably, the system includes a vacuum valve and a vacuum pump, wherein the vacuum valve is in communication with the vacuum chamber, and the vacuum pump acts on the vacuum chamber through the vacuum valve for maintaining a negative pressure state in the vacuum chamber.

Preferably, the system includes a vacuum purge valve in the vacuum chamber for completing a vacuum break of the vacuum chamber.

Preferably, the vacuum chamber is provided with and connected to a vacuum gauge, and the vacuum gauge is communicatively connected to the controller for acquiring a vacuum condition of the vacuum chamber.

Preferably, the system includes a display communicatively connected to the controller to display a status of the system.

The present invention also provides a microneedle fabrication method using the microneedle casting system described above, including the steps of: S1: placing a microneedle casting mold on the motion platform, closing the vacuum chamber, and evacuating the vacuum chamber and maintaining a vacuum state of the vacuum chamber; S2: driving, by the second drive component, the liquid-filling needle assembly to move to a designated position in the second direction, and filling a liquid into the liquid-filling needle assembly; meanwhile, driving, by the motion platform, the microneedle casting mold to move in the first direction or/and the third direction, and stopping filling the liquid when casting on the microneedle casting mold is completed; and S3: restoring the vacuum chamber to a normal pressure, opening a chamber door of the vacuum chamber, and removing the filled microneedle casting mold.

Preferably, the microneedle casting system includes the display communicatively connected to the controller, the filling pump communicatively connected to the liquid-filling needle shaft, the mixing tank communicatively connected to the filling pump, the vacuum valve and the vacuum gauge communicatively connected to the vacuum chamber, and the vacuum pump and the vacuum purge valve communicatively connected to the vacuum valve, wherein the filling pump, the vacuum gauge, the vacuum pump, the vacuum purge valve, and the vacuum valve are all communicatively connected to the controller, the method including the steps of: S11: setting process parameters on the display, adding raw materials for solution preparation into the mixing tank, placing the microneedle casting mold on the motion platform after mixing is completed, and closing the vacuum chamber; S21: clicking on the display to start a casting program, opening the vacuum valve, and evacuating the vacuum chamber with the vacuum pump; S31: stopping the vacuum pump when the vacuum gauge detects that a vacuum degree in the vacuum chamber reaches a first set value, and meanwhile, closing the vacuum valve to maintain a vacuum state in the vacuum chamber; S41: driving, by the second drive component, the liquid-filling needle assembly to move in the second direction to the designated position, starting to fill the liquid, by the filling pump, into the liquid-filling needle assembly; meanwhile, driving, by the motion platform, the microneedle casting mold to move in the first direction or/and the third direction, and stopping the filling pump and resetting the motion platform and the liquid-filling needle assembly to an initial position after the casting on the microneedle casting mold is completed; and S51: opening the vacuum purge valve to restore the vacuum chamber to the normal pressure, opening the chamber door of the vacuum chamber, and removing the filled microneedle casting mold.

The present invention is more advantageous than the prior art in that according to the microneedle casting system and microneedle fabrication method provided in the present invention, a filling liquid can be applied evenly on a large-area plane of a casting mold in the case of a wide range of filling amounts by configuring a motion platform of a microneedle casting mold in a vacuum chamber, to achieve high-precision and rapid replication of a micro-nano structure, consuming less casting solution and showing high casting efficiency. A combination of the vacuum chamber, a liquid-filling needle, and the motion platform renders the casting of solutions with different viscosities possible while keeping a high negative pressure in the vacuum chamber, and after casting, the pressure difference ensures that all the solutions can flow into the micro-nano structure beneath the surface of the mold, hence the accuracy and consistency of the mold replication is guaranteed. In particular, a pressure-reducing valve is configured in the liquid-filling needle to continuously spray the liquid in a wide range when filling with solutions having different viscosities and ensure that a small amount of casting solution is applied evenly on a surface of the mold, which is more advantageous than other casting methods. The vacuum chamber is connected to a vacuum pump and a vacuum gauge, and the vacuum chamber can be evacuated before or during filling with the solution so that a high negative pressure state is maintained in the vacuum chamber and the casting solution flows into the micro-nano structure beneath the surface of the mold to remove residual gases in the micro-structures, which ensures the accuracy of mold replication. The whole casting process is controlled by a control system, with less liquid consumption and high efficiency.

### BRIEF DESCRIPTION OF THE DRAWINGS

FIG. 1 is a schematic view showing an overall structure of a microneedle casting system according to an embodiment of the present invention;

FIG. 2 is a schematic view showing a structure of a vacuum chamber according to an embodiment of the present invention;

FIG. 3 is a schematic view showing a partial structure of a motion platform and a liquid-dispensing tip of a liquid-filling needle according to an embodiment of the present invention;

FIG. 4 is a schematic view showing a partial structure of the motion platform and the liquid-dispensing tip of the liquid-filling needle according to another embodiment of the present invention;

FIG. 5 is a schematic view showing a partial cross-section of the liquid-dispensing tip of the liquid-filling needle according to an embodiment of the present invention;

FIG. 6 is a schematic view showing a cross-section of a pressure-reducing valve of the liquid-filling needle according to an embodiment of the present invention;

FIG. 7 is a bottom view of the pressure-reducing valve of the liquid-filling needle according to an embodiment of the present invention;

FIG. 8 is a schematic view showing an overall structure of the pressure-reducing valve according to an embodiment of the present invention;

FIG. 9 is a schematic view showing a cross-section of the pressure-reducing valve according to an embodiment of the present invention;

FIG. 10 is a schematic view showing a structure of a piston of the pressure-reducing valve according to an embodiment of the present invention;

FIG. 11 is a schematic view showing a structure of a diaphragm of the pressure-reducing valve according to an embodiment of the present invention;

FIG. 12 is a schematic view showing a structure of a microneedle casting mold according to an embodiment of the present invention.

### IN THE DRAWINGS

1-vacuum chamber, 2-vacuum pump, 3-controller, 4-display, 5-mixing tank, 6-filling pump, 7-first motion assembly, 8-motion platform, 9-second motion assembly, 10-vacuum gauge, 11-liquid-filling needle assembly, 12-vacuum valve, 13-vacuum purge valve, 14-microneedle casting mold, 71-first motor, 72-first transmission component, 73-connecting shaft, 74-first coupling, 75-second coupling, 711-output end, 721-support frame, 722-first guide rod, 723-second guide rod, 724-moving member, 725-first screw, 726-second screw, 727-third screw, 728-connecting gear set, 91-second motor, 92-vertical post, 93-sliding rail, 94-slider, 11-1 liquid-dispensing tip, 11-2 pressure relief valve, 11-3 pressure-reducing valve, 11-4 liquid-filling needle shaft, 11-30, valve body, 11-31 piston, 11-32 diaphragm, 11-33 inflow channel, 11-34 inflow hole, 11-35 outflow hole, 11-36 damping hole, 11-37 outflow channel, 11-38 external flow hole, 11-300 cavity channel, 11-301-first securing component, 11-302-second securing component, 11-391 first compression spring, 11-392 second compression spring, 11-310-piston body, 11-311 first cavity, 11-312 piston seat, 11-313 first stop post, 11-314 first projection, 11-315-ramp, 11-320-diaphragm body, 11-321 second cavity, 11-322 diaphragm seat, 11-323 second stop post, 11-324 second projection, 11-371 back-flow hole, 101-side wall, 102-top wall, 141-concave cavity, 142-microneedle needle body concave hole.

### DETAILED DESCRIPTION OF THE INVENTION

In order that the object, aspects, and advantages of the invention will become more apparent, a detailed description of the invention will be rendered by reference to the appended drawings and embodiments.

This embodiment provides a microneedle casting system including a vacuum chamber 1, a vacuum pump 2, a controller 3, a display system 4, a mixing tank 5, a filling pump 6, a first motion assembly 7, a motion platform 8, a second motion assembly 9, a vacuum gauge 10, a liquid-filling needle assembly 11, a vacuum valve 12, and a vacuum purge valve 13.

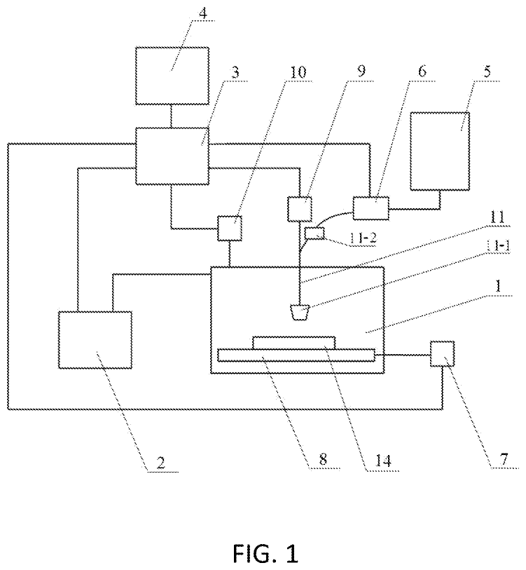

As shown in FIG. 1, the vacuum chamber 1 is configured to provide a closed environment to facilitate the fabrication of microneedles under a vacuum condition. The motion platform 8 is provided in the vacuum chamber 1. In this embodiment, the motion platform 8 is capable of a unidirectional motion for supporting the microneedle casting mold 14. As shown in FIG. 8, the microneedle casting mold 14 includes a concave cavity 141 for fabricating the microneedle, the concave cavity 141 has a bottom surface provided densely with microneedle needle body concave holes 142, and the shape, size, arrangement pattern, and density of the microneedle needle body concave holes 142 match the microneedles to be fabricated. In this embodiment, the vacuum condition for fabricating the microneedles is not specifically limited, and a person skilled in the art can determine the details of the vacuum condition according to the properties of the materials for fabricating the microneedles, the structure, density, and size of the microneedles, etc.

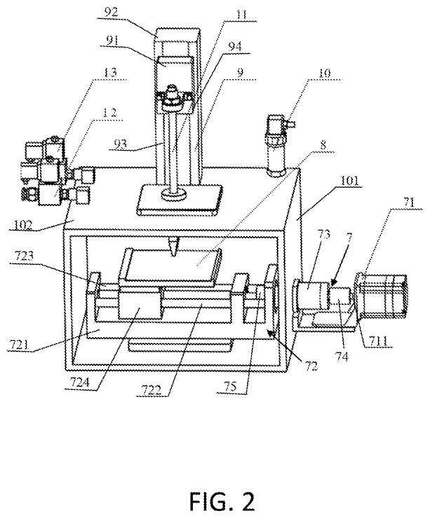

As shown in FIGS. 1 to 3, the first motion assembly 7 is communicatively connected to the controller 3 for driving the motion platform 8 to move in a first direction. In this embodiment, the first direction is the left-right direction in FIG. 2. Further, the first motion assembly 7 includes a first drive component and a first transmission component 72. The first drive component is a first motor 71, which in this embodiment performs a rotational movement. The first transmission component 72 serves to convert the rotational movement of the first motor 71 into a linear movement of the motion platform 8 in the first direction. Further, the first transmission component 72 includes a support frame 721, a first guide rod 722, a second guide rod 723, a moving member 724, and a first screw 725. The first guide rod 722 and a second guide rod 723 are provided on the support frame 721 in the first direction, and the first guide rod 722 and the second guide rod 723 pass through the moving member 724. The first screw 725 is rotatably arranged on the support frame 721 in the first direction. The moving member 724 is in transmission connection with the first motor 71, and the first screw 725 is in a threaded connection with the moving member 724. The motion platform 8 is fixed to the moving member 724 for moving the microneedle casting mold 14. Specifically, when an output end 711 of the first motor 71 performs a rotational movement, the first screw 725 is driven to rotate. Since the moving member 724 is in a threaded connection with the first screw 725, and under the constraint of the first guide rod 722 and the second guide rod 723, the moving member 724 can be driven to perform a linear movement in the first direction, and then the motion platform 8 fixed on the moving member 724 is driven to perform a linear movement in the first direction. More specifically, the first motor 71 is outside the vacuum chamber 1, the first transmission component 72 is inside the vacuum chamber 1, and the first motor 71 and the first transmission component 72 are connected through a connecting mechanism. The connecting mechanism includes a connecting shaft 73, a first coupling 74, and a second coupling 75. One end of the connecting shaft 73 is connected to the output end 711 of the first motor 71 through the first coupling 74, and the other end of the connecting shaft 73 passes through a side wall 101 of the vacuum chamber 1 between the first drive component and the first transmission component and is connected to the first screw 725 through the second coupling 75. A bearing and a sealing ring are provided between the connecting shaft 73 and the side wall 101 of the vacuum chamber 1 between the first drive component and the first transmission component, to enable a sealed rotary connection. In some alternative embodiments, the first motion assembly 7 is configured to move the motion platform 8 in the first direction and the third direction, wherein the first direction and the third direction are perpendicular to each other, for example, the first direction is a left-right direction in FIG. 2, and the third direction is perpendicular to the plane of the figure; the present invention is not particularly limited in this regard, and a person skilled in the art can configure the motion platform 8 to be driven to move horizontally in one direction or multiple directions as appropriate. In other embodiments, the first motor 71 may be inside the vacuum chamber 1, and the first motor 71 may be a stepper or servo motor, etc. In this embodiment, there are two guide rods, namely, the first guide rod 722 and the second guide rod 723. In alternative embodiments, there may be one or more than two guide rods.

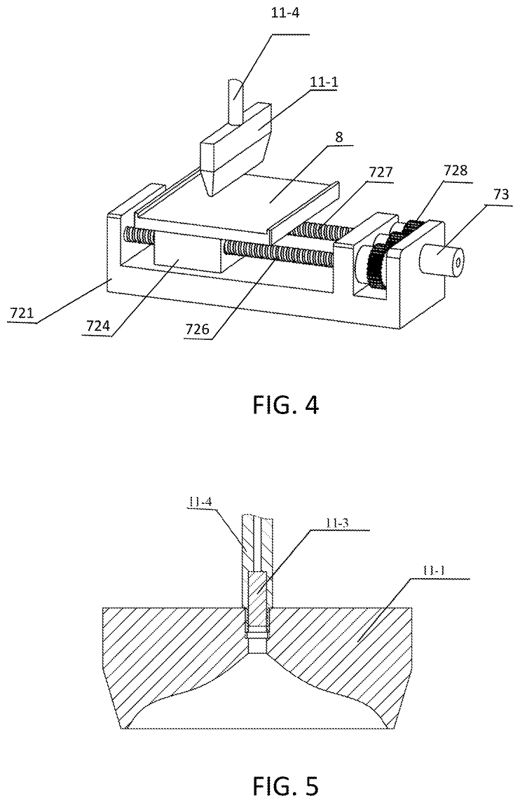

In another embodiment, as shown in FIG. 4, the first transmission component 72 includes the support frame 721, a second screw 726, a third screw 727, and the moving member 724. The second screw 726 and the third screw 727 are rotatably arranged on the support frame 721 in the first direction and are in a threaded connection with the moving member 724 for driving the moving member 724 to move in the first direction. For example, the second screw 726 and the third screw 727 are provided with external threads, the moving member 724 is provided with internal threads, and the external threads of the second screw 726 and the third screw 727 have the same pitch and direction. Specifically, when the output end 711 of the first motor 71 performs a rotary motion, the second screw 726 and the third screw 727 are driven to rotate, and since the moving member 724 is in a threaded connection with the second screw 726 and the third screw 727, the moving member 724 is driven to perform a linear motion in the first direction, and then the motion platform 8 fixed on the moving member 724 is driven to perform a linear motion in the first direction. More specifically, the first motor 71 is outside the vacuum chamber 1, the first transmission component 72 is inside the vacuum chamber 1, and the first motor 71 and the first transmission component 72 are connected through the connecting mechanism. The connecting mechanism includes the connecting shaft 73, the first coupling 74, and a connecting gear set 728. One end of the connecting shaft 73 is connected to the output end 711 of the first motor 71 through the first coupling 74, and the other end of the connecting shaft 73 passes through the side wall 101 of the vacuum chamber 1 between the first drive component and the first transmission component and is connected to the second screw 726 and the third screw 727 through the connecting gear set 728. The second screw 726 has a second thread that is in a threaded connection with the moving member, and the third screw has a third thread that is in a threaded connection with the moving member 724. The second screw 726 and the third screw 727 are driven to rotate in the same direction, and the second thread has an identical pitch and direction to those of the third thread; alternatively, the second screw 727 and the third screw 728 may be driven to rotate in opposite directions, and the second thread has an identical pitch to but different direction from that of the third thread.

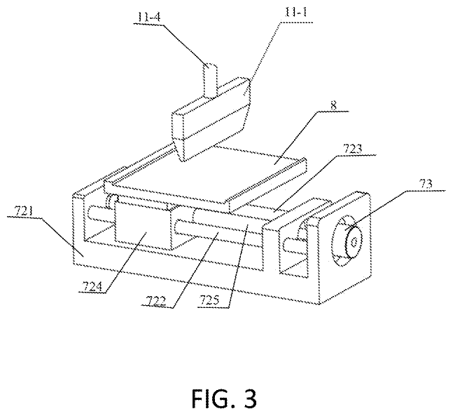

With continued reference to FIGS. 1, 2, and 5, the liquid-filling needle assembly 11 includes a liquid-filling needle shaft 11-4, a filling pump 6, and a liquid-dispensing tip 11-1. The liquid-filling needle assembly 11 is configured to deliver a casting solution for fabricating microneedles into the vacuum chamber 1. Specifically, the liquid-filling needle shaft 11-4 includes a first end and a second end. The first end of the liquid-filling needle shaft 11-4 passes through a top wall 102 of the vacuum chamber 1, enters the interior of the vacuum chamber 1, and is connected to the liquid-dispensing tip 11-1. The second end of the liquid-filling needle shaft 11-4 is connected to the filling pump 6 through a hose, and the filling pump 6 may be a plunger pump, a screw pump, a peristaltic pump, etc. Preferably, the liquid-filling needle assembly 11 further includes a pressure-reducing valve 11-3. The pressure-reducing valve 11-3 is provided inside the liquid-filling needle shaft 11-4 and can realize one or more of the functions of reducing pressure, stabilizing pressure, and back-flowing of the casting solution. The pressure-reducing valve 11-3 is optionally in a threaded connection with or secured to the liquid-filling needle shaft 11-4. More preferably, as shown in FIGS. 3, 4, and 5, the liquid-dispensing tip 11-1 is flattened, the liquid-dispensing tip 11-1 is detachably connected to the liquid-filling needle shaft 11-4 or they are an integral structure, the liquid-dispensing tip 11-1 has a liquid outlet port extending in a direction perpendicular to the third direction, and the cross-section of the liquid outlet port of the liquid-dispensing tip 11-1 can be square, rectangular, receptacle-shaped, trumpet-shaped, and the like. The liquid-dispensing tip 11-1 can be sized according to the condition of the casting solution. Preferably, a size of the liquid-dispensing tip 11-1 extending in the direction perpendicular to the third direction is smaller than a size of the microneedle casting mold 14 in the third direction so that the solution can be sprayed to cover a wide range. More preferably, the liquid outlet port of the liquid-dispensing tip 11-1 has a length of 1 cm to 40 cm and a width of 0.05 mm to 5 mm.

As shown in FIG. 2, the liquid-filling needle assembly 11 is driven by the second motion assembly 9 to move towards or away from the motion platform 8 in the second direction. The second direction is perpendicular to both the first direction and the third direction. In this embodiment, the second direction is the vertical direction in FIG. 2. The second motion assembly 9 includes a second drive component, which is a second motor 91, and a second transmission component, which includes a vertical post 92, a sliding rail 93, and a slider 94. The vertical post 92 can be placed on a frame or fixed on the top wall 102 of the vacuum chamber 1; the sliding rail 93 is arranged on the vertical post 92 in the second direction, one side of the slider 94 is movably connected to the sliding rail 93, and the other side of the slider 94 is fixedly connected to the liquid-filling needle shaft 11-4. Specifically, the sliding rail 93, driven by the second motor 91, moves the slider 94 in the second direction, thereby moving the liquid-filling needle assembly 11 in the second direction. The second motor 91 may be a stepper or servo motor or the like, and a range of elevation of the liquid-filling needle assembly 11 (e.g., the liquid-dispensing tip 11-1) in the second direction is preferably from 0 to 20 cm.

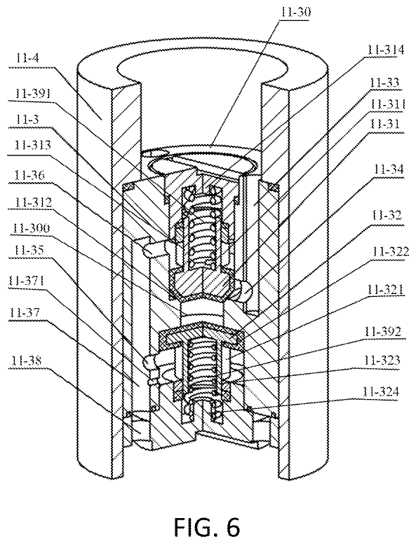

With reference to FIGS. 6, 8, and 9, in an embodiment, the pressure-reducing valve 11-3 includes a valve body 11-30 having an inner cavity formed therein, a first spool valve and a second spool valve are provided axially at an interval therein, and the first spool valve and the second spool valve are movable relative to the inner cavity. The first spool valve and the second spool valve may be pistons or diaphragms, and in this embodiment, the first spool valve is described in detail as a piston 11-31 and the second spool valve as a diaphragm 11-32. In other embodiments, the present invention is not particularly limited to such a configuration, for example, the first spool valve may be a diaphragm, the second spool valve may be a piston, or both the first spool valve and the second spool valve are pistons or diaphragms, as may be desired.

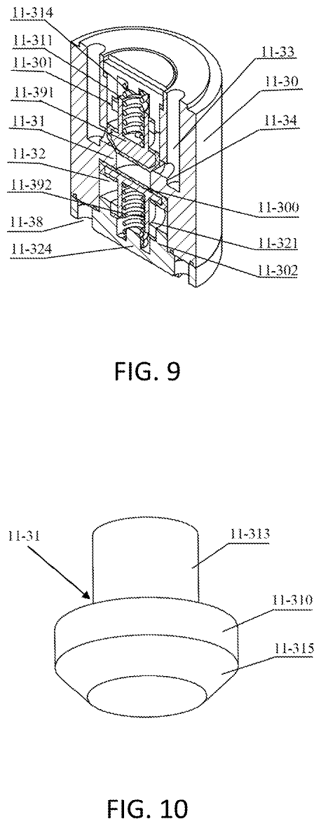

The valve body 11-30 is provided with an inflow channel 11-33, an inflow hole 11-34, an outflow hole 11-35, a damping hole 11-36, and an outflow channel 11-37. Herein, the inflow channel 11-33 is a blind hole for the inflow of the casting solution, and the inflow channel 11-33 is arranged to axially extend on the valve body 11-30. Preferably, the inflow channel 11-33 are plural, and a plurality of inflow channel 11-33 are uniformly distributed on the valve body 11-30 circumferentially. The number of the inflow channels 11-33 and the inflow holes 11-34 are not particularly limited in this embodiment, for example, the number can be one, two, four, five, six, eight, and ten. In the embodiment shown in FIGS. 8 and 9, the number of inflow channels 11-33 is two. An open end of the inflow channel 11-33 is closer to the second end of the liquid-filling needle shaft 11-4. The inflow hole 11-34 are provided in a cavity wall of the inner cavity for communicating the inflow channel 11-33 with the inner cavity, and the number of the inflow holes 11-34 equals the number of the inflow channels 11-33.

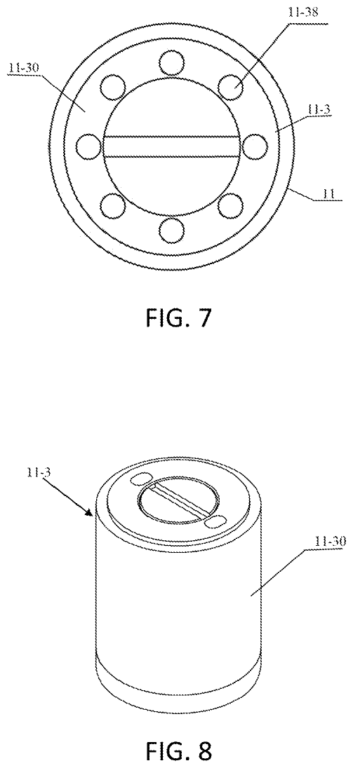

Similarly, the outflow channels 11-37 are blind holes for the outflow of the casting solution and are axially distributed on the valve body 11-30. Further, the pressure-reducing valve includes an external flow hole 11-38 communicating with the outflow channel 11-37. The open end of the outflow channel 11-37 is closer to the first end of the liquid-filling needle shaft 11-4. The outflow hole 11-35 is provided in the cavity wall of the inner cavity for communicating the outflow channel 11-37 with the inner cavity. The number of the outflow holes 11-35, the outflow channels 11-37, and the external flow holes 11-38 is not particularly limited in this embodiment, for example, the number can be one, two, four, five, six, eight, and ten. Preferably, a plurality of the outflow channels 11-37 are provided and uniformly distributed on the valve body 11-30 circumferentially. The number of the outflow holes 11-35 and the external flow holes 11-38 may equal the number of outflow channels 11-37, in which case outlets of the outflow channels 11-37 may be communicated directly with the external flow holes 11-38. The number of the external flow holes 11-38 may not equal the number of the outflow channels 11-37, and preferably, the number of the external flow holes 11-38 is greater than the number of the outflow channels 11-37 so that the casting solution can flow out quickly. In the embodiment shown in FIG. 7, the number of the outflow channels 11-37 is two and the number of the external flow hole 11-38 is eight, in which case, an annular recess is further provided between the outflow channel 11-37 and the external flow hole 11-38 so that the outflow channel 11-37 is communicated with the external flow hole 11-38. A plurality of the external flow holes 11-38 are uniformly distributed circumferentially. According to the positions of the inflow hole 11-34 and the outflow hole 11-35, the inner cavity is divided into a first cavity 11-311, a cavity channel 11-300, and a second cavity 11-321 successively. In this embodiment, the inner cavity includes a third end and a fourth end. The third end is closer to the second end of the liquid-filling needle shaft 11-4 than the fourth end. The inner cavity between the third end and the inflow hole 11-34 is the first cavity 11-311, the inner cavity between the inflow hole 11-34 and the outflow hole 11-35 is the cavity channel 11-300, and the inner cavity between the outflow hole 11-35 and the fourth end is the second cavity 11-321.

The first spool valve 11-31 is configured to, initially, hold at a first position under a first elastic force and abut against the inflow hole 11-34 to cut off communication between the cavity channel 11-300 and the inflow channel 11-33; move axially from the first position to the first cavity 11-311 against the first elastic force under a first axial pressure of the casting solution greater than the first elastic force to communicate the cavity channel 11-300 with the inflow channel 11-33. The first spool valve 11-31 is further configured to return to the first position under the first elastic force when the first axial pressure of the casting solution is less than the first elastic force or the first axial pressure of the casting solution is removed, and abut against the inflow hole 11-34 to cut off the communication between the cavity channel 11-300 and the inflow channel 11-33. In a preferred embodiment, the first position is located where the inflow hole 11-34 is communicated with the cavity channel 11-300. The diaphragm 11-32 is configured such that, initially, the diaphragm 11-32 is held in a second position under a second elastic force so as to cut off communication between the cavity channel 11-300 (between the piston 11-31 and the diaphragm 11-32) and the outflow channel 11-37; when the diaphragm 11-32 is subjected to a second axial pressure of the casting solution greater than the second elastic force, the diaphragm 11-32 moves in an axial direction of the inner cavity from the second position to the second cavity 11-321 against the second elastic force, so that the cavity channel 11-300 (between the piston 11-31 and the diaphragm 11-32) is communicated with the outflow channel 11-37. Also, when the second axial pressure of the casting solution that is less than the second elastic force or the second axial pressure of the casting solution is removed, the diaphragm 11-32 is returned to the second position by the second elastic force to prevent the cavity channel 11-300 (between the piston 11-31 and the diaphragm 11-32) from being communicated with the outflow channel 11-37. In a preferred embodiment, the second position is between the first position and the outflow hole 11-35. The first axial pressure and the second axial pressure may be equal or not.

In this embodiment, as shown in FIGS. 6 and 9, the piston 11-31 is above the diaphragm 11-32. Accordingly, the first cavity 11-311 is above the cavity channel 11-300, and the cavity channel 11-300 is above the second cavity 11-321. The pressure-reducing valve 11-3 further includes a first secured member 11-301 and a second secured member 11-302. The first secured member 11-301 is secured at an end of the third end of the inner cavity; the second secured member 11-302 is secured at an end of the fourth end of the inner cavity. Accordingly, the first cavity 11-311 is defined by the first secured member 11-301 and the inflow hole 11-34; the second cavity 11-321 is defined by the second secured member 11-302 and the outflow hole 11-35.

The pressure-reducing valve 11-3 further includes a first valve seat and a second valve seat. The first valve seat is configured to maintain the first spool at the first position and prevent the first spool from approaching the second spool; the second valve seat is adapted to maintain the second spool valve at the second position and prevent the second spool valve from approaching the first spool valve. In this embodiment, the first position is a position where the inflow hole 11-34 and the cavity channel 11-300 are communicated; the second position is between the first position and the outflow hole 11-35. The first valve seat is a piston seat 11-312; the second valve seat is a diaphragm seat 11-322. The piston seat 11-312 serves to prevent the piston 11-31 from approaching the diaphragm 11-32; the diaphragm seat 11-322 serves to prevent the diaphragm 11-32 from approaching the piston 11-31. Specifically, the piston seat 11-312 is a first step provided on the valve body 11-30, shaped to match the shape of the piston 11-32; the diaphragm seat 11-322 is a second step provided on the valve body 11-30, shaped to match the shape of the diaphragm 11-32. Further, as shown in FIG. 10, the piston 11-31 has a ramp 11-315 so that the first force applied to the piston 11-31 by the casting solution may have an axial component.

With reference also to FIGS. 6, 9, and 10, a first elastic structure, such as a first compression spring 11-391, is provided within the first cavity 11-311 for providing the first elastic force. Specifically, one end of the first compression spring 11-391 abuts against the piston 11-31, and the other end of the first compression spring 11-391 abuts against the first secured member 11-301. Further, the piston 11-31 includes a piston body 11-310 for abutting against an end of the inflow channel 11-33 at the side of the cavity channel 11-300 at the first position to cut off the communication between the cavity channel 11-300 and the inflow channel 11-33. The piston body 11-310 is provided with a hollow first stop post 11-313 extending towards the first secured member 11-301 in addition to the ramp 11-315. An inner diameter of the first stop post 11-313 is slightly larger than that of the first compression spring 11-391 to receive the first compression spring 11-391 and prevent the first compression spring 11-391 from moving, sloshing, or twisting radially. Further, the first secured member 11-301 is provided with a first recess for receiving the first stop post 11-313. Further, at a bottom of the first recess, a first projection 11-314 is formed by extending towards the piston 11-31. An end of the first compression spring 11-391 is sleeved outside the first projection 11-314 to further prevent the first compression spring 11-391 from moving, sloshing, or twisting radially. Initially, a certain distance exists between an open end of the first stop post 11-313 and the bottom of the first recess to ensure a space in which the piston 11-31 can move axially.

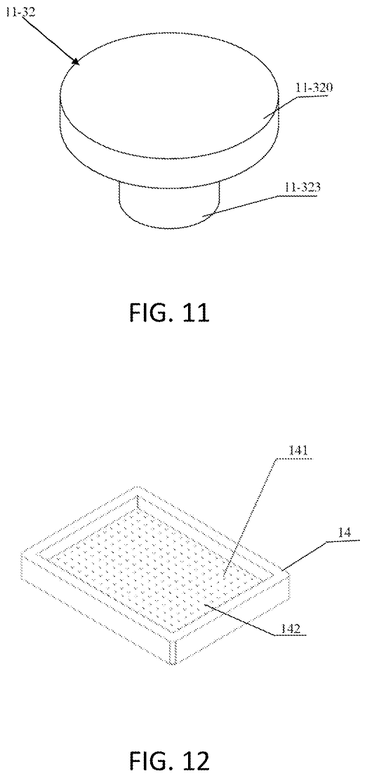

With reference also to FIGS. 6, 9, and 11, a second elastic structure, such as a second compression spring 11-392, is arranged within the second cavity 11-321 for providing a second elastic force. Specifically, one end of the second compression spring 11-392 abuts against the diaphragm 11-32, and the other end of the second compression spring 11-392 abuts against the second secured member 11-302. Further, the diaphragm 11-32 includes a diaphragm body 11-320 provided with a hollow second stop post 11-323 extending towards the second secured member 11-302. An inner diameter of the second stop post 11-323 is slightly larger than that of the second compression spring 11-392 to accommodate the second compression spring 11-392 and prevent the second compression spring 11-392 from moving, sloshing, or twisting in radially. Further, the second secured member 11-302 is provided with a second recess for receiving the second stop post 11-323. Even further, at a bottom of the second recess, a second projection 11-324 is formed by extending towards the diaphragm 11-32. An end of the second compression spring 11-392 is sleeved outside the second projection 11-324 to further prevent the second compression spring 11-392 from moving, sloshing, or twisting radially. Initially, a certain distance exists between an open end of the second stop post 11-323 and the bottom of the second recess to ensure a space in which the diaphragm 11-32 can move axially.

Further, a damping hole 11-36 is provided on a cavity wall of the first cavity 11-311, and the outflow channel 11-37 and the first cavity 11-311 are communicated through the damping hole 11-36. The damping hole 11-36 serves to ensure that the pressure in the first cavity 11-311 between the piston 11-31 and the first secured member 11-301 is maintained stable when the piston 11-31 moves. Preferably, a distance between the damping hole 11-36 and the first position is greater than the distance between the open end of the first stop post 11-313 and the bottom of the first recess to prevent the piston 11-31 from blocking the damping hole 11-36.

Further, a back-flow hole 11-371 through which the outflow channel 11-37 is communicated with the second cavity 11-321 is provided in a cavity wall of the second cavity 11-321. The back-flow hole 11-371 serves to discharge the casting solution flowing back into the second cavity 11-321. When the diaphragm 11-32 is reset to the second position, the casting solution flows back into the inner cavity defined by the diaphragm 11-32 and the second secured member 11-302, and, if not discharged in time, will cause the diaphragm 11-32 to fail to move towards the second secured member 11-302, hence the back-flow hole 11-371 in the cavity wall of the second cavity 11-321 is necessary. Preferably, the back-flow hole 11-371 is positioned at an end of the second secured member 11-302 near the diaphragm 11-32.

When the pressure-reducing valve 11-3 of this embodiment is in use, the casting solution enters the inflow hole 11-34 through the inflow channel 11-33 of the pressure-reducing valve 11-3, and exerts the first force on the piston 11-31; when the first force cancels the first elastic force, the piston 11-31 moves to the first cavity 11-311 in a direction towards the first secured member 11-301, the inflow hole 11-34 is communicated with the cavity channel 11-300, and the casting solution enters the cavity channel 11-300; the casting solution in the cavity channel 11-300 then applies a second force to the diaphragm 11-32; when the second force is greater than the second elastic force, the diaphragm 11-32 moves to the second cavity 11-321 in a direction towards the second secured member 11-302, the outflow hole 11-35 is communicated with the cavity channel 11-300, and the casting solution enters the outflow channel 11-37 and finally flows out from the external flow hole 11-38. After the delivery of the casting solution to the pressure-reducing valve 11-3 is stopped, when the second force on the diaphragm 11-32 is less than the second elastic force, the diaphragm 11-32 moves towards the piston 11-31 and holds at the second position to cut off the communication between the cavity channel 11-300 (between the piston 11-31 and the diaphragm 11-32) and the outflow hole 11-35; when the first force applied to the piston 11-31 is less than the first elastic force, the piston 11-31 moves towards the diaphragm 11-32 and holds at the first position to cut off the communication between the inflow hole 11-34 and the cavity channel 11-300.

In this embodiment, a pressure-reducing function of the casting solution is achieved by balancing the axial pressure of the solution with the elastic force of the first elastic structure and the second elastic structure, in conjunction with an internal throttling function of the pressure-reducing valve 11-3; it is also possible to prevent the casting solution from shooting out from the liquid-filling needle shaft 11-4 in a vacuum environment. Further, the outflow channel 11-37 is in communication with the damping hole 11-36, so that when the piston 11-31 moves towards the first secured member, the pressure inside the first cavity 11-311 is discharged through the outflow channel 11-37; the pressure of the external flow hole 11-38 is fed back to the first cavity 11-311 and then to the piston 11-31 through the outflow channel 11-37; therefore, the arrangement of the damping hole 11-36 stabilizes the output pressure of the casting solution when the piston 11-31 reciprocates axially. When the casting is stopped, and after the pressure of the casting liquid in the casting pipeline is removed, the piston 11-31 and the diaphragm 11-32 are reset to the first position and the second position, respectively, under the action of the first elastic force and the second elastic force, and the solution flows back through the outflow hole 11-35 at the same time of resetting to avoid liquid drops at the liquid outlet of the liquid-filling needle, which greatly reduces the liquid drops caused by the repeated evacuation during the repeated liquid filling of the liquid-filling needle assembly and improves the liquid filling accuracy and the quality of the microneedle.

More preferably, a pressure relief valve 11-2 is connected between the liquid-filling needle shaft 11-4 and the filling pump 6, and the pressure relief valve 11-2 is connected to the liquid-filling needle shaft 11-4 and the filling pump 6, respectively, through a hose. In addition, the pressure relief valve 11-2 is configured to be switched on and off in response to signals that are opposite, but in complete synchronization with, those for the filling pump 6. Once the liquid-filling needle 11 completes one time of filling, the filling pump 6 is switched off while the pressure relief valve 11-2 is switched on to remove the fluid pressure in the liquid-filling needle shaft 11-4. At this time, the piston 11-31 and the diaphragm 11-32 are reset to their initial positions under the action of the first elastic force and the second elastic force; meanwhile, the solution quantitatively flows back, which greatly reduces the liquid drops when the liquid-filling needle assembly 11 is used for repetitive liquid filling and improves the accuracy of filling liquid and the quality of the microneedle.

The mixing tank 5 is configured to mix various raw materials for fabricating the microneedles uniformly to form a casting solution for fabricating the microneedles. The mixing tank 5 can mix and stir materials; more preferably, the mixing tank 5 can enable material dispersion, homogenization, emulsification, etc. The filling pump 6 is communicatively connected to the controller 3 and connected to the mixing tank 5 to pump the mixed casting solution to the liquid-dispensing tip 11-1. In this way, the mixing tank 5 can continuously feed the filling pump 6 to complete a continuous casting in batch.

A vacuum valve 12 is provided in the vacuum chamber 1 to open or close a vacuum pipeline. The vacuum pump 2 acts on the vacuum chamber 1 through the vacuum valve 12. The vacuum pump 2 is communicatively connected to the controller 3. The vacuum pump 2 is configured to evacuate the vacuum chamber 1 before and/or during the filling of the solution, so as to maintain a negative pressure state in the vacuum chamber 1. After filling, because of the pressure difference between the interior and exterior of the microneedle casting mold 14, the casting solution can flow into the microneedle needle body concave hole 142 beneath the surface of the microneedle casting mold 14, which ensures the accuracy of the microneedle casting mold 14 replication. The vacuum pump 2 may be an oil pump or a dry pump.

A vacuum gauge 10 is communicatively connected to the controller 3 and is provided in the vacuum chamber 1 for detecting a vacuum condition of the vacuum chamber 1, such as a degree of vacuum, and feeding back the vacuum condition to the controller 3. A vacuum purge valve 13 is provided in the vacuum chamber 1 for breaking the vacuum of the vacuum chamber 1. The vacuum pump 2 cooperates with the vacuum gauge 10, and the degree of vacuum of the vacuum chamber 1 is controlled by the controller 3 generally. Preferably, the vacuum valve 12 is an electronic vacuum valve, and the vacuum purge valve 13 is an electronic vacuum purge valve, both controlled by the controller 3.

A display 4 serves as an input/output device of the system and is communicatively connected to the controller 3 for receiving instructions from the outside and displaying the status of the system. The controller 3 controls the whole operation of the microneedle casting system centrally. The controller 3 is communicatively connected to the first drive component and the second drive component, and is configured to control the second drive component to move the liquid-filling needle assembly towards the motion platform in the second direction when the vacuum condition is present in the vacuum chamber 1, to control the liquid-filling needle assembly to deliver the microneedle fabrication material into the vacuum chamber, and to control the first drive component to move the motion platform in the first direction or/and the third direction. The controller 3 controls the first motor 71, the second motor 91, the filling pump 6, and the vacuum pump 2 to realize the unified and coordinated operation of the whole microneedle casting system. This embodiment is not limiting as to of which particular type the controller 3 is, and the so-called controller may be a central processing unit (CPU) or other general-purpose processors, a digital signal processor (DSP) 301, an application-specific integrated circuit (ASIC), a field-programmable gate array (FPGA) or other programmable logic devices, a discrete gate or transistor logic device, a discrete hardware component, etc. The general-purpose processor may be a microprocessor or any conventional processor, etc. The processor is a control center of an electronic device, and all the parts of the whole electronic device are connected through various interfaces and lines.

The present invention also provides a microneedle fabrication method using the microneedle casting system described above, including the steps of:

S1: placing the microneedle casting mold 14 on the motion platform 8, closing the vacuum chamber 1, evacuating the vacuum chamber 1, and maintaining a vacuum state of the vacuum chamber;

S2: driving, by the second drive component, the liquid-filling needle assembly to move to a designated position in the second direction, and filling a liquid into the liquid-filling needle assembly 11-4; meanwhile, driving, by the motion platform, the microneedle casting mold to move in the first direction or/and the third direction, stopping filling the liquid when casting on the microneedle casting mold 14 is completed, and returning the motion platform 8 and the liquid-filling needle assembly 11-4 to their initial positions; and

S3: restoring the vacuum chamber 1 to a normal pressure, opening a chamber door of the vacuum chamber 1, and removing the filled microneedle casting mold 14.

Specifically, when using the above devices, the operator sets process parameters through the user interface of the display 4, and then adds the raw materials for fabricating the microneedles to the mixing tank 5. After the materials are mixed, the microneedle casting mold 14 is placed on the motion platform 8, and the chamber door of the vacuum chamber 1 is closed. The operator clicks on the display 4 to start the casting process, the vacuum valve 12 is opened, and the vacuum pump 2 evacuates the vacuum chamber 1. When the vacuum gauge 10 detects that the vacuum degree of the vacuum chamber 1 reaches a first set value, the vacuum pump 2 is stopped while the vacuum valve 12 is automatically closed to maintain the vacuum state of the vacuum chamber 1. Subsequently, the second motion assembly 9 moves the liquid-filling needle assembly 11 to a designated position, the filling pump 6 starts filling, and at the same time, the motion platform 8 moves the microneedle casting mold 14. Preferably, when the vacuum gauge 10 detects that the vacuum degree of the vacuum chamber 1 is lower than a second set value, the vacuum valve 12 is opened, and the vacuum pump 2 evacuates the vacuum chamber 1 until the vacuum degree of the vacuum chamber 1 reaches the first set value. When the casting for the microneedle casting mold 14 is completed, the filling pump 6 is stopped, and the motion platform 8 and the liquid-filling needle assembly 11 are reset to their initial positions. After this, the vacuum purge valve 13 is opened, the vacuum chamber 1 is returned to the normal pressure, the chamber door of the vacuum chamber 1 is opened, and the filled microneedle casting mold 14 is removed, that is, a single round of casting is completed. The process can be repeated for operation in batch.

Therefore, according to the microneedle casting system provided herein, casting a mold with a large-area plane in a high vacuum environment is possible, highly accurate and rapid replication of a micro-nano structure is enabled, only a small amount of casting solution is consumed, and high casting efficiency is rendered. The advantages lie in at least that:

the microneedle casting system is provided with a mixing tank capable of continuously feeding to the filling pump to complete continuous casting in batch; the end of the filling pump is connected to and inserted into the liquid liquid-liquid-filling needle assembly in the vacuum chamber, and the liquid liquid-liquid-filling needle assembly can have the functions of vacuum drip prevention and liquid-discharge pressure reducing, allowing for accurate filling under the condition of a high vacuum degree; the end of the liquid-filling needle shaft is connected to the flat liquid-dispensing tip, and the flattened liquid outlet port with a wide width can render a continuous spray of liquid in a wide range; in conjunction with the motion platform for placing the microneedle casting mold, an even distribution over the plane of the microneedle casting mold can be achieved in the case of different filling amounts; the vacuum chamber is connected to the vacuum pump and the vacuum gauge, and the vacuum chamber is evacuated before or during filling of the solution to maintain a high negative pressure state in the vacuum chamber, hence the casting solution flows into the micro-nano structures beneath the surface of the microneedle casting mold, and the residual gas in the micro-nano structures is removed to ensure the accuracy for the microneedle casting mold replication. The whole casting process is controlled centrally by the control system, with less liquid consumption and higher efficiency.

Although the present invention has been described with reference to the preferred embodiments, it is not intended to be limited by the embodiments. Those skilled in the art can make some modifications and improvements without departing the scope of the present invention. The scope of the present invention is defined by the claims.

1. A microneedle casting system, comprising: a vacuum chamber, a motion platform, a first motion assembly, a liquid-filling needle assembly, a second motion assembly, and a controller; wherein
the motion platform is arranged in the vacuum chamber for supporting a microneedle casting mold;
the first motion assembly comprises a first transmission component and a first drive component that are connected to each other, the motion platform is connected to the first transmission component, and the first transmission component, driven by the first drive component, moves the motion platform in a first direction or/and a third direction;
the liquid-filling needle assembly is configured to convey a casting solution for fabricating the microneedle into the vacuum chamber, the liquid-filling needle assembly comprises a liquid-dispensing tip and a liquid-filling needle shaft, one end of the liquid-filling needle shaft extends into the vacuum chamber and is connected to the liquid-dispensing tip;
the second motion assembly comprises a second transmission component and a second drive component that are connected to each other, the liquid-filling needle shaft is connected to the second transmission component, and the second transmission component, driven by the second drive component, moves the liquid-filling needle assembly in a second direction;
the controller is communicatively connected to the first drive component and the second drive component, respectively, the controller is configured, when the vacuum chamber is vacuum, to control the second drive component to drive the liquid-filling needle assembly to move closer to the motion platform in the second direction and to control the liquid-filling needle assembly to output the casting solution for fabricating the microneedle, and to control the first drive component to drive the motion platform to move in the first direction and/or the third direction;
wherein the first direction, the second direction, and the third direction are perpendicular to each other.

2. The microneedle casting system according to claim 1, wherein the first drive component is a first motor, the first motor having an output end connected to the first transmission component; the first transmission component comprises a support frame, a guide rod, a first screw, and a moving member, where the first screw is rotatably arranged on the support frame in the first direction, the guide rod is arranged in the first direction and passes through the moving member, the moving member is in a threaded connection with the first screw and moves in the first direction under the action of the guide rod and the first screw, and the motion platform is fixedly connected to the moving member.

3. The microneedle casting system according to claim 2, wherein the first drive component is outside the vacuum chamber, the first transmission component is inside the vacuum chamber, and the first drive component and the first transmission component are connected through a connecting mechanism; the connecting mechanism comprises a connecting shaft, a first coupling, and a second coupling, where one end of the connecting shaft is connected to an output end of the first motor via the first coupling, and the other end of the connecting shaft passes through a side wall of the vacuum chamber and the support frame and is connected to the first screw through the second coupling; the connecting shaft is sealed and rotatably arranged on the side wall of the vacuum chamber between the first drive component and the first transmission component.

4. The microneedle casting system according to claim 1, wherein the first drive component is a first motor, the first motor having an output end connected to the first transmission component; the first transmission component comprises a support frame, a second screw, a third screw, and a moving member, where the second screw and the third screw are rotatably arranged on the support frame in the first direction, the moving member moves in the first direction by being threadedly connected to the second screw and the third screw, and the motion platform is fixedly connected to the moving member.

5. The microneedle casting system according to claim 4, wherein the second screw has a second thread for a threaded connection with the moving member, the third screw has a third thread for a threaded connection with the moving member, the second screw and the third screw are driven to rotate in one direction, and the second thread has an identical pitch and direction to those of the third thread; alternatively,
the second screw and the third screw are driven to rotate in opposite directions, and the second thread has an identical pitch to but a different direction from that of the third thread.

6. The microneedle casting system according to claim 4, wherein the first drive component is outside the vacuum chamber, the first transmission component is inside the vacuum chamber, and the first drive component and the first transmission component are connected through a connecting mechanism; the connecting mechanism comprises a connecting shaft and a connecting gear set, one end of the connecting shaft is connected to an output shaft of the first motor, the other end of the connecting shaft passes through a side wall of the vacuum chamber and the support frame and is connected to the second screw and the third screw through the connecting gear set; the connecting shaft is sealed and rotatably arranged on the side wall of the vacuum chamber between the first drive component and the first transmission component.

7. The microneedle casting system according to claim 3, wherein a bearing and a sealing ring are provided between the connecting shaft and the side wall of the vacuum chamber between the first drive component and the first transmission component, so that the connecting shaft is sealed and rotatably arranged on the side wall of the vacuum chamber between the first drive component and the first transmission component.

8. The microneedle casting system according to claim 1, wherein the second drive component is a second motor, and the second transmission component comprises a vertical post, a sliding rail, and a slider, where the vertical post is fixedly arranged outside the vacuum chamber, the sliding rail is arranged on the vertical post in the second direction, one side of the slider is movably connected to the sliding rail, and the other side of the slider is fixedly connected to the liquid-filling needle shaft; the sliding rail, driven by the second motor, moves the liquid-filling needle assembly in the second direction.

9. The microneedle casting system according to claim 1, wherein the liquid-filling needle assembly further comprises a pressure-reducing valve within the liquid-filling needle shaft for performing one or more functions of pressure reducing, pressure stabilizing, and back flowing of the casting solution.

10. The microneedle casting system according to claim 9, wherein the pressure-reducing valve is at an end of the liquid-filling needle shaft close to the liquid-dispensing tip, and the pressure-reducing valve is provided with an external thread for a threaded connection with the liquid-filling needle shaft; the end of the liquid-filling needle shaft close to the liquid-dispensing tip is provided with an external thread for a threaded connection with the liquid-dispensing tip.

11. The microneedle casting system according to claim 9, comprising a valve body, where an inner cavity is formed in the valve body, a first spool valve and a second spool valve are provided in the inner cavity at an interval axially, and the first spool valve and the second spool valve are movable relative to the inner cavity; an inflow channel, an inflow hole, an outflow hole, and an outflow channel are provided on the valve body, the inflow channel and the outflow channel are blind holes, and the inflow channel and the outflow channel extend axially on the valve body;
the inflow channel is in communication with the inner cavity through the inflow hole, the outflow channel is in communication with the inner cavity through the outflow hole, and according to positions of the inflow hole and the outflow hole, the inner cavity is divided into a first cavity, a cavity channel, and a second cavity successively;
the first spool valve is configured to, initially, hold at a first position under a first elastic force and abut against the inflow hole to cut off communication between the cavity channel and the inflow channel; move axially from the first position to the first cavity against the first elastic force under a first axial pressure of the casting solution greater than the first elastic force to communicate the cavity channel with the inflow channel;
the second spool valve is configured to, initially, hold in a second position under a second elastic force to cut off communication between the cavity channel between the first spool valve and the second spool valve and the outflow channel; move axially along the inner cavity from the second position to the second cavity against the second elastic force under a second axial pressure of the casting solution greater than the second elastic force, to communicate the cavity channel between the first spool valve and the second spool valve with the outflow channel.

12. The microneedle casting system according to claim 11, wherein the first spool valve is further configured to return to the first position under the first elastic force when the first axial pressure of the casting solution is less than the first elastic force or the first axial pressure of the casting solution is removed;
the second spool valve is further configured to return to the second position under the second elastic force when the second axial pressure of the casting solution is less than the second elastic force or the second axial pressure of the casting solution is removed.

13. The microneedle casting system according to claim 11, wherein a cavity wall of the first cavity is further provided with a damping hole through which the outflow channel is communicated with the first cavity.

14. The microneedle casting system according to claim 11, wherein the pressure-reducing valve further comprises a first secured member and a second secured member, the inner cavity has a third end and a fourth end, the first secured member is secured to an end of the third end of the inner cavity, and the second secured member is secured to an end of the fourth end of the inner cavity;
a first elastic structure is provided in the first cavity for providing the first elastic force, one end of the first elastic structure abuts the first spool valve, and the other end of the first elastic structure abuts the first secured member;
a second elastic structure is arranged within the second cavity for providing the second elastic force, one end of the second elastic structure abuts the second spool valve, and the other end of the second elastic structure abuts the second secured member.

15. The microneedle casting system according to claim 14, wherein the first spool valve comprises a first spool valve body having a hollow first stop post extending axially within the first cavity, and the first elastic structure is arranged within the first stop post; the second spool valve comprises a second spool valve body having a hollow second stop post extending axially within the second cavity, and the second elastic structure is arranged within the second stop post.

16. The microneedle casting system according to claim 15, wherein the first secured member is further provided with a first recess for receiving the first stop post; the second secured member is further provided with a second recess for receiving the second stop post.

17. The microneedle casting system according to claim 16, wherein a first projection is formed by extending the first recess towards the first spool valve, and the other end of the first elastic structure is sheathed outside the first projection; a second projection is formed by extending the second recess towards the second spool valve, and the other end of the second elastic structure is sheathed outside the second projection.

18. The microneedle casting system according to claim 17, wherein a cavity wall of the first cavity is further provided with a damping hole through which the outflow channel is communicated with the first cavity;
a distance between the damping hole and the first position is greater than a distance between an open end of the first stop post and a bottom of the first recess.

19. The microneedle casting system according to claim 11, wherein the valve body is provided with a first valve seat and a second valve seat, where the first valve seat is configured to maintain the first spool valve at the first position and prevent the first spool valve from approaching the second spool valve, and the second valve seat is configured to maintain the second spool valve at the second position and prevent the second spool valve from approaching the first spool valve.

20. The microneedle casting system according to claim 11, wherein the valve body is further provided with a back-flow hole through which the outflow channel is communicated with the inner cavity, the back-flow hole being located between the outflow hole and an outlet of the outflow channel.

21. The microneedle casting system according to claim 20, wherein the first spool valve is a piston and the second spool valve is a diaphragm, the piston having a ramp for generating the first axial pressure of the casting solution against the piston.

22. The microneedle casting system according to claim 1, wherein the liquid-filling needle assembly further comprises a filling pump, one end of the liquid-filling needle shaft away from an end of the liquid-dispensing tip being connected to the filling pump, the filling pump being communicatively connected to the controller.

23. The microneedle casting system according to claim 22, further comprising a mixing tank connected to the filling pump for mixing various raw materials for fabricating the microneedle uniformly to form the casting solution for fabricating the microneedle.

24. The microneedle casting system according to claim 22, wherein the other end of the liquid-filling needle shaft is connected to a pressure relief valve, and the pressure relief valve is connected to the liquid-filling needle shaft and the filling pump through hoses, respectively, for relieving liquid pressure in the liquid-filling needle shaft.

25. The microneedle casting system according to claim 1, comprising a vacuum valve and a vacuum pump, wherein the vacuum valve is in communication with the vacuum chamber, and the vacuum pump acts on the vacuum chamber through the vacuum valve for maintaining a negative pressure state in the vacuum chamber.

26. The microneedle casting system according to claim 1, comprising a vacuum purge valve in the vacuum chamber for completing a vacuum break of the vacuum chamber.

27. The microneedle casting system according to claim 1, wherein the vacuum chamber is provided with and connected to a vacuum gauge, and the vacuum gauge is communicatively connected to the controller for acquiring a vacuum condition of the vacuum chamber.

28. The microneedle casting system according to claim 1, comprising a display communicatively connected to the controller to display a status of the system.

29. A microneedle fabrication method, using the microneedle casting system according to claim 1, comprising the steps of:
S1: placing a microneedle casting mold on the motion platform, closing the vacuum chamber, evacuating the vacuum chamber, and maintaining a vacuum state of the vacuum chamber;
S2: driving, by the second drive component, the liquid-filling needle assembly to move to a designated position in the second direction, and filling a liquid into the liquid-filling needle assembly; meanwhile, driving, by the motion platform, the microneedle casting mold to move in the first direction or/and the third direction, and stopping filling the liquid when casting on the microneedle casting mold is completed; and
S3: restoring the vacuum chamber to a normal pressure, opening a chamber door of the vacuum chamber, and removing the filled microneedle casting mold.

30. The microneedle fabrication method according to claim 29, wherein the microneedle casting system comprises the display communicatively connected to the controller, the filling pump communicatively connected to the liquid-filling needle shaft, the mixing tank communicatively connected to the filling pump, the vacuum valve and the vacuum gauge communicatively connected to the vacuum chamber, and the vacuum pump and the vacuum purge valve communicatively connected to the vacuum valve, where the filling pump, the vacuum gauge, the vacuum pump, the vacuum purge valve, and the vacuum valve are all communicatively connected to the controller, the method comprising the steps of:
S11: setting process parameters on the display, adding raw materials for solution preparation into the mixing tank, placing the microneedle casting mold on the motion platform after mixing is completed, and closing the vacuum chamber;
S21: clicking on the display to start a casting program, opening the vacuum valve, and evacuating the vacuum chamber with the vacuum pump;
S31: stopping the vacuum pump when the vacuum gauge detects that a vacuum degree in the vacuum chamber reaches a first set value, and meanwhile, closing the vacuum valve to maintain a vacuum state in the vacuum chamber;
S41: driving, by the second drive component, the liquid-filling needle assembly to move in the second direction to the designated position, starting to fill the liquid, by the filling pump, into the liquid-filling needle assembly; meanwhile, driving, by the motion platform, the microneedle casting mold to move in the first direction or/and the third direction, and stopping the filling pump and resetting the motion platform and the liquid-filling needle assembly to an initial position after the casting on the microneedle casting mold is completed; and
S51: opening the vacuum purge valve to restore the vacuum chamber to the normal pressure, opening the chamber door of the vacuum chamber, and removing the filled microneedle casting mold.
